(19) United States
(12) Patent Application Publication
(10) Pub. No.: US 2026/0183787 A1
(43) Pub. Date: Jul. 2, 2026

(54) VIBRATING MESH MODULE WITH INTEGRATED FLOW CHAMBER

(71) Applicant: Philip Morris Products S.A., Neuchatel (CH)

(72) Inventors: Dara Zaman BAYAT, Neuchâtel (CH); Leander DITTMANN, Crissier (CH); Dino FARALLI, Grandson (CH); Laura GALLIMBERTI, Morges (CH)

(73) Assignee: Philip Morris Products S.A., Neuchatel (CH)

(21) Appl. No.: 19/129,336

(22) PCT Filed: Dec. 14, 2023
(86) PCT No.: PCT/EP2023/085784

§ 371 Date: May 13, 2025

(30) Foreign Application Priority Data
Dec. 14, 2022 (EP) 22213497.5

(51) Int. Cl. B05B 17/00; B05B 17/06

(52) U.S. Cl. CPC B05B 17/0646; B05B 17/0676

(57) ABSTRACT

An aerosol-generating device including a vibrating mesh module for an aerosol-generating device is provided, the vibrating mesh module including: a MEMS membrane with a plurality of through-holes defining perforations of the MEMS membrane; a flow chamber located adjacent to the MEMS membrane, the flow chamber defining a volume configured to hold an aerosol-forming substrate and to feed the aerosol-forming substrate to the MEMS membrane, the flow chamber being configured to include an inlet through which the aerosol-forming substrate flows into the volume of flow chamber, and an outlet through which the aerosol-forming substrate may exit the volume of the flow chamber, the flow chamber being manufactured using MEMS techniques; a liquid storage portion in fluid communication with the inlet and the outlet of the flow chamber of the vibrating mesh module; and a pumping device configured to generate a liquid flow through the integrated cavity.

The present invention relates to a vibrating mesh module for use in an aerosol-generating device. The present invention also relates to an aerosol-generating device comprising such vibrating mesh module. The present invention also relates to a method of manufacturing a membrane for a vibrating mesh module for use in an aerosol-generating device.

Aerosol-generating devices using vibrating mesh modules are often also referred to as vibrating mesh (VM) nebulizers. Such vibrating mesh nebulizers are used for the generation of a respirable aerosol that can be used for instance for treating respiratory diseases.

Vibrating mesh nebulizers use mesh deformation or vibration to push a liquid through a mesh. In a typical vibrating mesh nebulizer, a piezo element, which is in contact with a mesh, is used to produce vibrations of the mesh. The mesh is adjacent and in direct contact with a liquid drug-containing substrate. Holes in the mesh may have a conical structure, with the largest cross-section of the cone in contact with the liquid substrate. The mesh deformation generates a pressure field in the liquid, thus pumping and loading the holes with liquid. The liquid volume displaced through the holes breaks up into droplets, and are ejected into an mouthpiece chamber. The droplets mix with the air and directly form an aerosol in the mouthpiece chamber. During inhalation, ambient air crosses the mouthpiece chamber to transport the aerosol to the user.

Several off-the-shelves vibrating mesh nebulizer devices are available today. These devices comprise vibrating mesh modules that may mainly consist of two parts: An annular piezo-element and a circular, perforated membrane. In case the membrane is formed from metal or stainless steel, the perforations are typically created by laser drilling. Laser drilling leads to conical holes having a small cone angle and a comparably high aspect ratio. Membranes being made from nickel or a nickel alloy, are typically perforated by a combined lithography and electroplating process. In a first step, such membranes are perforated using a lithographic process to obtain a desired pattern of holes with a comparatively large diameter. Then the entire membrane undergoes electroplating to deposit material on the surface thereby shrinking the size of holes. The shape of the resulting holes is funnel-like, which is generally more preferred with respect to their microfluidic properties. In addition, this process may result in a lower aspect ratio and is therefore also for this reason preferred over conically shaped laser drilled holes.

The size of the holes of the membranes used in either of these commercially available devices is limited to about 3 to 4 micrometers. Therefore, the MMAD (Median Mass Aerodynamic Diameter) of the aerosol size distribution obtainable by such devices is also in the range of 3 to 4 micrometers, which is too big for deeper lung inhalation. Accordingly, most of the aerosol will be deposited in the upper region of the respiratory tract, which may be disadvantageous for efficient drug uptake and may cause throat irritation.

Reduction of the dimension of the holes in conventional membranes may not be possible, since a decreasing hole diameter requires an increased pressure to push the liquid through the hole. This increase could be compensated by reducing the aspect ratio (length of the hole divided by diameter of the hole) of the holes. However, due to mechanical stability reasons the thickness of the membranes cannot be arbitrarily reduced in conventional manufacturing methods. Further, the liquid throughput rate per hole decreases quadratically with decreasing hole diameter. Thus, in order to maintain a desired aerosol throughput such membranes would have to be provided with a significantly higher number of holes.

Leakage is also critical when liquid substrates are used for generation of aerosol. Leakage may occur if the liquid substrate in the supply portion is pressurized or if for other reasons more liquid substrate is dispensed than can be aerosolized by the aerosolization unit of the aerosol-generating device.

In order to allow for reliable and reproducible aerosol formation, it is necessary to ensure that the membrane, more specifically the entrance of the holes in the membrane, is always in contact to a homogeneous liquid layer while the VM module is operated to generate aerosol.

It would therefore be desirable to provide a VM module which ensures that the membrane is sufficiently supplied with aerosol-forming substrate. It would further be desirable to at the same time prevent leakage of aerosol-forming substrate from the aerosol-generating device.

It would further be desirable to provide a perforated membrane for a vibrating mesh module overcoming at least one of the abovementioned drawbacks.

It would be desirable to provide a perforated membrane allowing to generate aerosol particles having a reduced diameter while at the same time maintaining the volume of the generated aerosol.

It would further be desirable to provide a manufacturing method that allows to reliably and reproducibly manufacture perforated membranes with specific aerosol generating properties. In particular it would be desirable to provide a method to manufacture vibrating mesh modules without the need of glue or a gluing step.

In summary, there is a need in the art to manufacture membranes for VM modules with reduced exit hole diameter, decreased aspect ratio of the through holes, increased number of holes, and avoiding glue to assemble the module.

According to an embodiment of the invention there is provided a vibrating mesh module for use in an aerosol-generating device. The vibrating mesh module comprises a MEMS membrane with a plurality of through-holes to define perforations of the membrane and a flow chamber located adjacent to the MEMS membrane. The flow chamber defines a volume configured for holding aerosol-forming substrate and for feeding the aerosol-forming substrate to the MEMS membrane.

As used herein a “MEMS membrane” is a membrane suitable to be used in a vibrating mesh module, wherein the membrane is manufactured by micro-electro-mechanical systems (MEMS) processing techniques as described in more detail herein below.

The perforated MEMS membrane comprises an entrance side, which faces towards the flow chamber. The perforated MEMS membrane further comprises a release side, through which the aerosol-forming substrate is released into a mouthpiece chamber of the aerosol-generating device. The flow chamber of the vibrating mesh module may be configured such that the liquid aerosol-forming substrate flows through the volume of the flow chamber and in contact with the entrance side of the MEMS membrane.

By providing a flow chamber adjacent to the entrance side of the MEMS membrane, it is ensured that the membrane, more specifically the entrance of the through-holes in the membrane, is always in contact to a portion of the liquid substrate while the vibrating mesh module is operated to generate aerosol.

The flow chamber of the vibrating mesh module may be configured to comprise an inlet through which the liquid aerosol-forming substrate may flow into the volume of flow chamber and wherein the flow chamber is further configured to comprise an outlet through which aerosol-forming substrate may exit the volume of the flow chamber.

By configuring the flow chamber of the vibrating mesh module to not only comprise an inlet, but to also comprise an outlet, any excess aerosol-forming substrate may leave the flow chamber through its outlet, a pressure increase in the flow chamber due to excess supply of aerosol-forming substrate may be avoided. On the one hand this ensures that a homogenous pressure level is maintained in the flow chamber. On the other hand, this configuration reduces the risk that excess aerosol-forming substrate is dispensed through the membrane into the mouthpiece chamber of the aerosol-generating device.

The outlet of the flow chamber may be configured to have a flow resistance that is smaller than the flow resistance through the through-holes of the membrane. The flow resistance through the membrane is mainly determined by cross sectional dimension and the aspect ratio of the through-holes. The flow resistance of the outlet is mainly determined by its flow cross section. By suitable configuring the cross section of the outlet of the flow chamber, a desired low flow resistance may be established. The smaller the flow resistance of the outlet, the lower the risk that excess liquid substrate is accidentally dispensed through the membrane. In conventional vibrating mesh nebulizers, the membranes may be supplied with liquid substrate by using wick feeding elements. In order to ensure that liquid substrate is permanently supplied to the membrane, such wick feeding elements need to be in forced contact with the membrane. Such contact leads to damping of the membrane vibration and may cause undue power consumption. In contrast thereto, in the vibrating mesh module described herein, the liquid substrate may flow freely through the flow chamber and the liquid substrate does not exert any disadvantageous pressure or mechanical force on the membrane. This allows for more homogeneous and reproducible liquid feeding to all through-holes in the membrane and results also in a lower dampening of the membrane. Thus, such vibrating mesh modules may show an increased aerosolization efficiency.

The vibrating mesh module may further comprise a piezo-electric actuator, configured to produce the vibrations of the MEMS membrane. The piezo-electric actuator, may be a conventional piezo-element formed from piezo-electric material. The piezo-element may be formed integrally with the remaining parts of the vibrating mesh module. The piezo-element may be formed integrally with the remaining parts of the vibrating mesh module using MEMS processing techniques. By integrally forming the piezo-element using MEMS processing techniques, the cumbersome and fault-prone step of gluing the piezo element to the vibrating mesh module may be avoided.

The invention also relates to an aerosol-generating device with a vibrating mesh module comprising a membrane as described herein. As used herein, the term “aerosol-generating device” refers to a device that interacts with an aerosol-forming substrate to generate an aerosol. An aerosol-generating device may interact with one or both of an aerosol-generating article comprising an aerosol-forming substrate, and a cartridge comprising an aerosol-forming substrate. An aerosol-generating device may comprise a housing, electric circuitry including a controller, a power supply and a plurality of sensors.

The aerosol-generating device may further comprise a liquid storage portion for storing and providing a supply of liquid substrate to be aerosolized. The liquid storage portion may be in fluid communication with the inlet and the outlet of the flow chamber of the vibrating mesh module.

The aerosol-generating device may be configured to generate a circular flow of liquid substrate from the liquid storage portion to the inlet of the flow chamber, towards the entrance side of the membrane in the flow chamber, through the outlet of the flow chamber and back to the liquid supply portion. For this purpose, the aerosol-generating device may comprise a pumping device. The pumping device may generate a liquid flow through the flow chamber of the vibrating mesh module.

The present invention also relates to a method of manufacturing a vibrating mesh module for use in an aerosol-generating device. The method comprises the steps of manufacturing a membrane from a bulk wafer of a first material using MEMS techniques, providing a flow chamber, and attaching the flow chamber to the membrane to form the vibrating mesh module.

The flow chamber structure may be manufactured using MEMS techniques or micromachining techniques. These techniques allow to precisely machine the flow chamber to the desired dimensions.

The flow chamber may be manufactured from a bulk wafer of a second material using MEMS techniques. The bulk wafer material used for manufacture of the flow chamber may be the same material as the bulk wafer used in manufacture of the membrane.

Attachment of the membrane to the flow chamber may be carried out by any suitable attachment method. These components may be attached by bonding or by gluing. In this case, gluing may be a suitable method, since the gluing spot is sufficiently remote from the vibrating membrane. Accordingly, it is not expected that gluing of these two components has any undesired impact on the vibrational behaviour of the membrane. In particular it may be assumed that in this case gluing does not alter the impedance of the membrane.

The method of manufacturing the membrane may comprise the steps of providing a bulk wafer of a first material, the bulk wafer having opposing first and second surfaces, depositing a cover layer of a second material onto the first surface of the bulk wafer, providing through-holes to the cover layer using MEMS manufacturing techniques, etching the second surface of the bulk wafer to define recesses therein, etching the second surface of the bulk wafer until the bulk wafer material is removed from the recesses, and cutting out the membrane.

MEMS manufacturing may offer the possibility to include piezo-elements during the manufacturing process to a vibrating mesh module. The piezo-elements may be deposited on the membrane module. By incorporating the piezo-elements already during manufacture of the membrane module, attachment of the piezo-elements to the membrane module is facilitated. In conventional manufacturing methods, the piezo-elements have to be glued to the membrane module, which may usually be a cumbersome and fault-prone manufacturing step.

Furthermore, growing the piezo-elements directly to the membrane module allows for a more target specific geometric design of the piezo-elements. The piezo-electric material may be deposited on certain pre-defined locations of the membrane module. Deposition of the piezo-electric material may be obtained by MEMS techniques, such as by sputtering or coating techniques. A mask may be used to achieve the desired lateral geometry of the piezo-electric elements. Alternatively, the desired lateral geometry may also be achieved by providing an overall layer of piezo-electric material and by subsequently removing piezo-electric material from positions where it is not needed. Such removal may again be achieved by masking and etching, or by mechanical removal.

The one or more piezo-elements may be deposited on a contact surface of the membrane structure. The contact surface of the membrane structure may be an annular portion located at a peripheral region of the membrane structure. If deemed to be helpful, the contact portion may also be defined at other surface areas of the membrane structure.

In principle, any material may be used that is suitable to manufacture piezo-elements. Such suitable piezo-electric materials may include lead zirconate titanite, zinc oxide, barium titanite, aluminum nitride, aluminum scandium nitride, lithium niobate, ferroelectric ceramics with perovskite structure and combinations thereof.

The membrane may be manufactured by making use of micro-electro-mechanical systems (MEMS) manufacturing techniques. Such manufacturing techniques include process technologies used in semiconductor device fabrication. These techniques include deposition of material layers, patterning by photolithography and etching of material in order to produce the required shapes.

Accordingly, the membrane may be made of material suitable to be processed by MEMS manufacturing techniques. The membrane may comprise a bulk layer formed from a material provided in the form of a wafer. The wafer material may be made of silicon, silicon oxide, silicon nitride, aluminium nitride, or combinations thereof.

The membrane structure may comprise a plurality of additional layers of material. These layers may be applied to the bulk wafer by thin film deposition techniques.

MEMS technology allows to tailor the hole geometry and shape to meet the needs required by the application. As discussed above, this includes in particular the diameter of the through-holes, which is a crucial parameter to adjust the resulting aerosol droplet size. MEMS technology also allows to define the aspect ratio of the through-holes, which is an important parameter to enable and control the microfluidic flow through the through-holes. As will be discussed in more detail below, the MEMS manufacturing process may include a plurality of photolithographic masking and etching steps. The lateral size of the mask defines the lateral placement and sizes of recesses and through-holes in the membrane structure and can easily be adapted to specific needs. The combination of etchant, wafer material, and wafer lattice orientation allows to define the cross-sectional shape of the etched recess and the depth of the resulting recesses. Subsequent etching steps thus allow the manufacturing of complex shapes of the recesses and through-holes of the membrane structure.

The method of manufacturing the membrane may comprise a plurality of manufacturing steps, which are also used in MEMS manufacturing and which may include processes such as deposition of material layers, patterning by photolithography and etching of material in order to produce the required shapes.

Thin film deposition may be achieved by physical vapour deposition (PVD) including techniques such as evaporation, magnetron sputtering or pulsed laser deposition (PLD). These techniques can be used to deposit one or more layers of material in a desired sequence onto the bulk wafer.

Photolithography is a well-known technique, in which light is used to produce minutely patterned thin films of suitable materials over a bulk substrate, to protect selected areas of the substrate during subsequent etching, deposition, or implantation operations. Typically, ultraviolet light is used to transfer a geometric design from an optical mask to a light-sensitive chemical (photoresist) coated on the substrate. The photoresist either breaks down or hardens where it is exposed to light. The patterned film is then created by removing the softer parts of the coating with appropriate solvents.

The etching steps might be accomplished by wet etching or dry etching or a combination thereof. Dry etching, for example reactive ion etching or plasma etching, may be preferred, since it may be used to create vertical edges in a substrate, independent of the crystallographic orientation of the substrate. Wet etching techniques may also be used. However, these techniques are usually anisotropic and the resulting patterns are not only defined by the previously applied mask, but may also depend on the lattice orientation. For example, silicon in <100> orientation with a KOH etching results in pyramidal shapes through the thickness of the membrane with an angle defined by the lattice plane orientation.

The bulk wafer may be made of any first material that is suitable to be processed by MEMS manufacturing techniques. The bulk wafer material may be made of silicon, silicon oxide, silicon nitride, aluminium nitride, or combinations thereof. The materials of the one or more additional layers applied to the bulk wafer may also be selected from any material that is suitable to be processed by MEMS manufacturing techniques. The materials of the one or more additional layers applied to the bulk wafer may be selected from silicon, silicon oxide, silicon nitride, aluminium nitride, or combinations thereof. In addition, the additional layers may be formed from materials having specific functional properties, such as selective etching stoppers or materials enhancing flow properties of the liquid substrate that is to be aerosolised.

In the method of manufacturing a membrane for a vibrating mesh module for use in an aerosol-generating device a bulk wafer of a first material such as silicon is provided. Wafer material is commercially available with different dimensions. The final membrane structure may have a thickness of 5 micrometers to 500 micrometers. The membrane may have a thickness of 10 micrometers to 400 micrometers. The membrane may have a thickness of 30 micrometers to 200 micrometers. The thickness of the membrane structure significantly determines mechanical behaviour of the membrane. Accordingly, the membrane thickness may be chosen depending on the targeted operation frequency and material properties of the membrane. As discussed in more detail below the final thickness of the membrane structure may be adjusted by a selectively etching step during the manufacturing method.

Deposition of the cover layer onto the first surface of the bulk wafer may be performed by any of the above physical vapour deposition (PVD) techniques. The cover layer may be formed from silicon dioxide or silicon nitride.

The cover layer may have a thickness of 0.1 to 10 micrometers. The cover layer may have a thickness of 0.2 to 3 micrometers. The cover layer may have a thickness of 0.3 to 1 micrometer. The thickness of the cover layer and the dimensions of the through-holes defined therein are decisive parameters to control the resulting MMAD (Median Mass Aerodynamic Diameter). Due to considerations with respect to microfluidic properties, small aspect ratios are desired. Thus, cover layers having a small thickness may be preferred. The final choice of the thickness may represent a trade-off with mechanical stability, which may pose a lower limit of the thickness of the cover layer. This lower limit may also depend on the lateral extension of the cover layer and the expected forces applied to the cover layer during usage.

Subsequently through-holes are provided to the cover layer using further MEMS manufacturing techniques. The through-holes through the cover layer may be obtained by a combination of photolithography and etching. For this purpose, the cover layer may be provided with a mask defining the size and position of the through-holes on the cover layer. In a subsequent etching step, the unmasked areas of the cover layer may be etched away, resulting in the cover layer being provided with a plurality of through-holes.

The through-holes formed in the recessed portions may have a diameter of between 0.1 and 4 micrometers. The through-holes formed in the recessed portions may have a diameter of between 0.2 and 3 micrometers.

In a further method step, the second surface of bulk wafer is treated to define recesses therein. These recesses may again be obtained by masking and subsequent etching of the bulk wafer material. Etching of the silicon bulk wafer may be carried out by using sulfur hexafluoride (SF6). Etching of the second surface of the bulk wafer is continued until the bulk waver material is removed from the recesses. The recesses in the second surface are preferably positioned such that they coincide with the positions of the through-holes provided in the cover layer. The combination of the bulk wafer and the cover layer forms a membrane structure having recesses with reduced thickness.

By etching the second surface of the bulk wafer, the final thickness of the membrane structure may be defined. The thickness of the membrane largely determines the vibrational characteristics of the membrane structure.

The two sides of the membrane may also be referred to as the “entrance side” and the “release side” of the membrane. In this regard, the side of the membrane comprising the recesses is the “entrance side”, which in use faces the liquid storage portion, and through which liquid enters into the through-holes. The other side of the membrane forms the “release side” of the membrane, which is the side through which the liquid droplets are released into a downstream mouthpiece chamber. Accordingly, the cover layer comprising the through-holes is provided at the release side of the membrane structure.

In a final step the membrane is cut out from the bulk wafer material. The membrane is cut out to the required dimensions as needed in the application. Typically, the size of the bulk wafers is significantly larger than the required dimension of a membrane, such that in the manufacturing method a plurality of membrane structures may be manufactured in parallel on a single bulk wafer. Such parallel processing allows to manufacture the membranes conveniently in high quantity.

One or more additional layers of material may be applied in manufacturing the membrane. For example, an additional layer may be deposited between the bulk wafer and the cover layer. This additional cover layer may be configured as a selective etching stopper layer. Such selective etching stopper layer may protect the cover layer on the first surface of the bulk wafer, when the recesses are formed at the second surface of the bulk wafer. The selective etching stopper layer may ensure that the etching process for forming the recesses at the second surface of the bulk wafer terminates when the etching fluid has removed the bulk wafer material from the recesses and has reached the selective etching stopper layer. Typically, this is achieved by forming the selective stopper layer from a material that does not dissolve upon contact with the etching agent used for etching the bulk wafer material. The selective etching stopper layer may subsequently be removed by using a different etching solvent.

In the above example, in which sulfur hexafluoride is used for etching the silicon bulk wafer, the selective etching stopper layer may be formed from any material that does not dissolve upon contact with sulfur hexafluoride. A suitable material in this regard is silicon dioxide. Silicon dioxide does not dissolve upon contact with sulfur hexafluoride, such that the vertical etching process of the bulk wafer will stop, when the etching fluid reaches the silicon dioxide layer. This silicon dioxide layer may later on be removed by another etching agent, such as Hydrogen fluoride (HF) or Trifluoromethane (CHF3).

The technique of using selective etching stopper layer may generally be used to provide the membrane structure with layers having a predefined thickness. In this way also the material defining the thickness of the membrane itself may be deposited as a layer of material being sandwiched between two layers of selective etching stopper material. In this way particularly thin membranes may be provided. In this way also membranes having a well-defined thickness may be provided.

Manufacturing the membrane by use of MEMS techniques allows to configure the membrane structure to have desired surface properties that may be beneficial for the aerosolization process. In particular the material properties of the surfaces forming the through-holes and the surfaces that may come into contact with the liquid substrate to be aerosolized may be designed to have such desirous surface properties.

For example, a layer of polycrystalline silicon may be provided below and directly adjacent the cover layer. In this way the entrance side of the through-holes of the cover layer is lined with the layer of polycrystalline silicon. Since polycrystalline silicon is more hydrophilic as compared to single crystalline silicon or silicon nitride, microfluidic flow through the through-holes in enhanced.

The individual method steps discussed above may also be carried out in a different sequence. The skilled person may vary the order of the individual manufacturing steps as deemed suitable.

Below, there is provided a non-exhaustive list of non-limiting examples. Any one or more of the features of these examples may be combined with any one or more features of another example, embodiment, or aspect described herein.

Features described in relation to one embodiment may equally be applied to other embodiments of the invention.

The invention will be further described, by way of example only, with reference to the accompanying drawings in which:

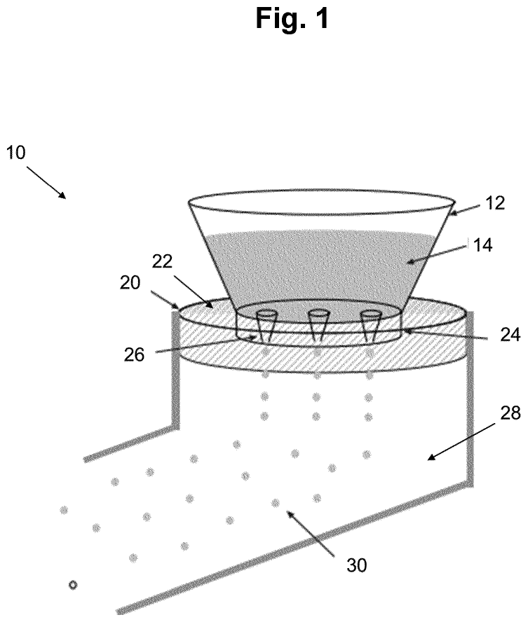

FIG. 1 shows a schematic design of a conventional vibrating mesh nebulizer;

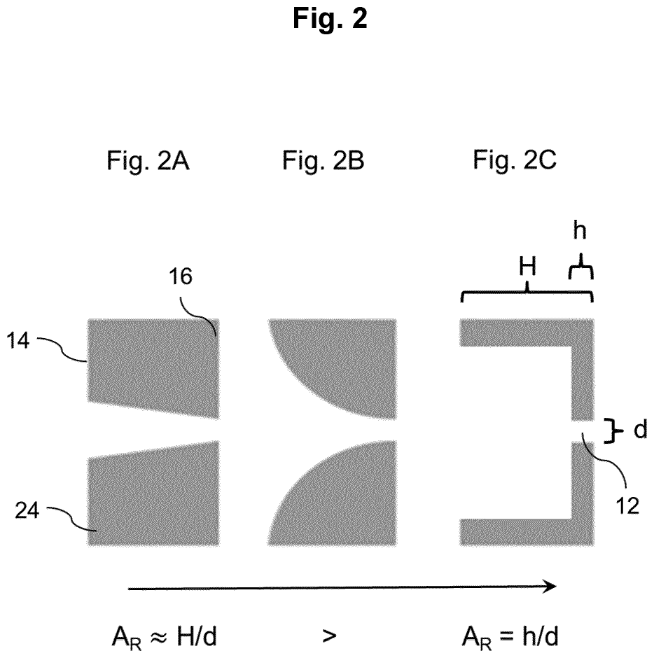

FIG. 2 shows geometries of through-holes in membranes obtainable by different manufacturing methods;

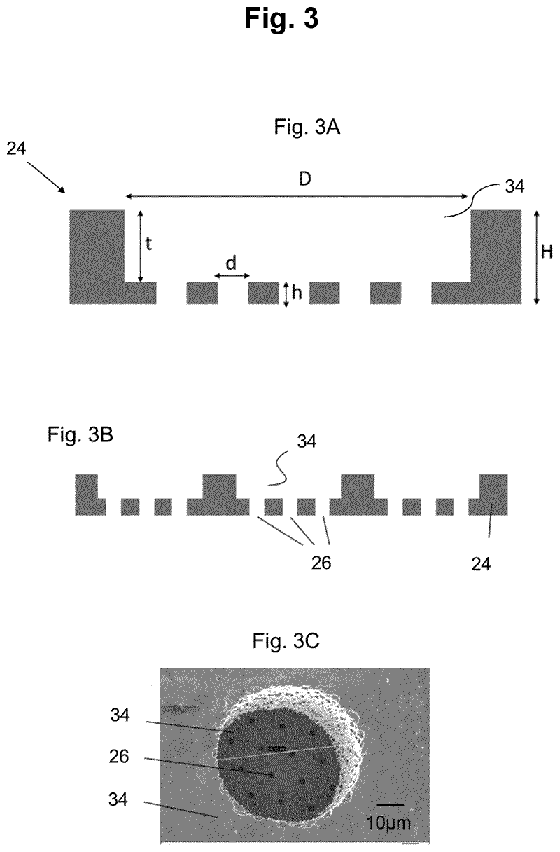

FIG. 3 shows detailed views of a MEMS membrane;

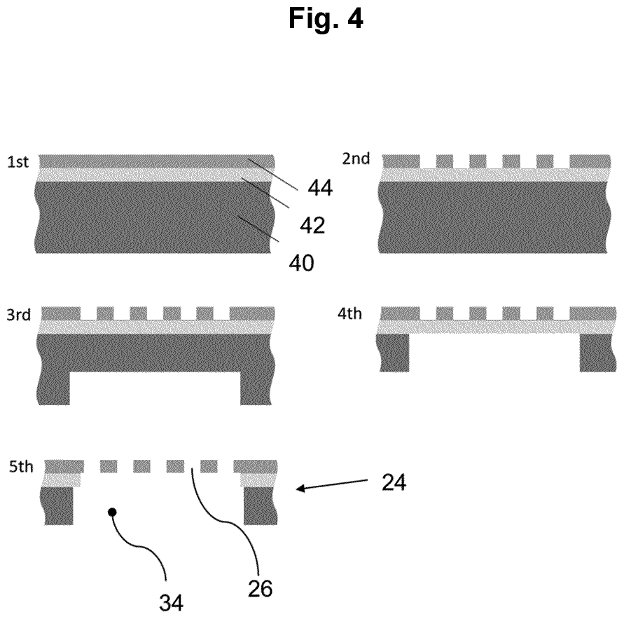

FIG. 4 illustrates the method steps to manufacture a MEMS membrane;

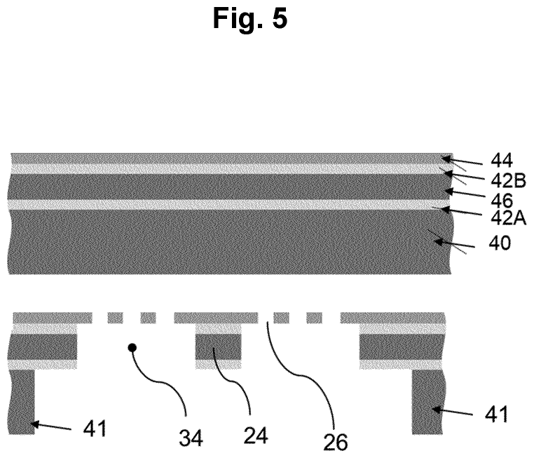

FIG. 5 shows a modification of the method of FIG. 4;

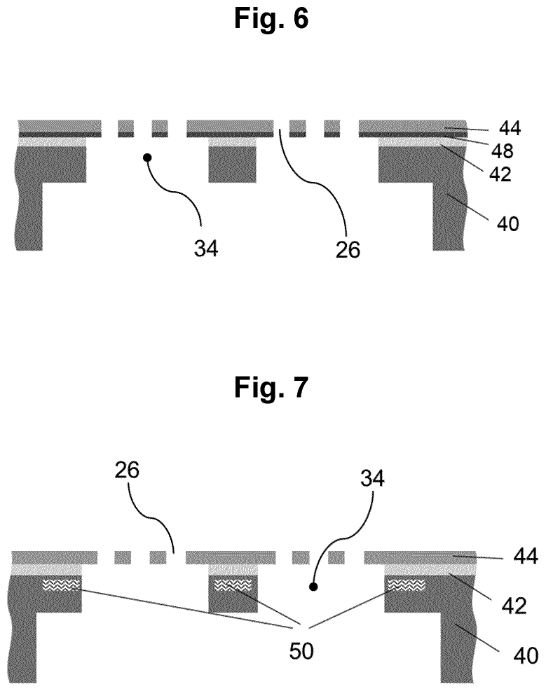

FIG. 6 shows a MEMS membrane provided with a hydrophilic layer;

FIG. 7 shows a MEMS membrane provided with an intrinsic heater;

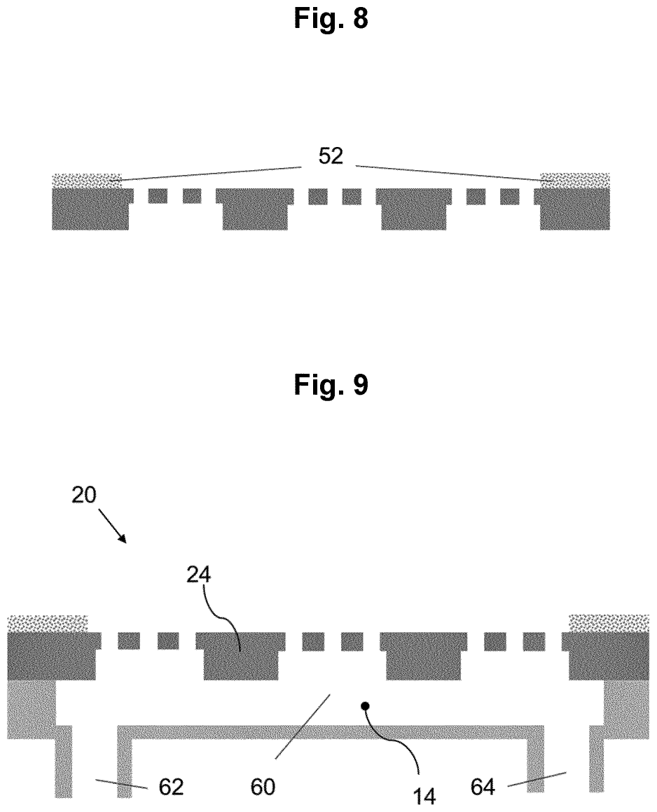

FIG. 8 shows a MEMS membrane provided with integrated piezo-elements;

FIG. 9 shows a VM module connected to an upstream liquid flow chamber; and

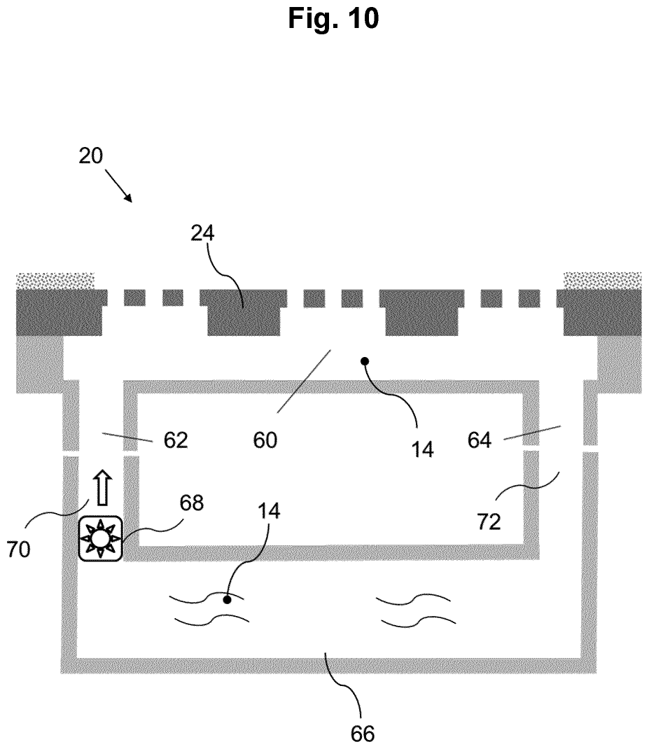

FIG. 10 shows the connection of the VM module to a liquid supply portion.

FIG. 1 shows an aerosol-generating device 10 that may also be referred to as a nebulizer. The nebulizer comprises a liquid storage portion 12 holding a supply of liquid substrate 14 to be aerosolized. In direct contact with the liquid substrate 14 there is provided a vibrating mesh module 20, which comprises an annular piezo-element 22 encompassing a circular mesh. The mesh is configured as a membrane 24 comprising through-holes 26. The through-holes 26 in the membrane 24 have a conical structure, with the largest cross-section of the cone in contact with the liquid drug. By vibration of the membrane 26 the liquid substrate 14 is pumped through the through-holes 26 and sprayed into a mouthpiece chamber 28. During inhalation, ambient air crosses the mouthpiece chamber 28 to transport the generated aerosol 30 to the user.

The size of the through-holes 26 at the exit side of the membrane 24 as well as the aspect ratio of the through-holes 26 are key parameters to determining the aerosolization process. FIG. 2 shows enlarged schematic views of membranes 24 comprising through-holes 26. The membranes 24 are depicted in descending order from left to right according to the aspect ratio AR of the through-holes 26 thereof. This order is indicated by the arrow pointing from the left hand side to the right hand side in FIG. 2. Each membrane 24 has an identical thickness H and the through-holes 26 have an identical minimum diameter d at the exit side 32 of the membrane 24. The through-holes 26 in the membranes 24 are obtained by different manufacturing methods, wherein each manufacturing method leads to through-holes 26 having different aspect ratios. In general, an aspect ratio AR of a through-hole 26 is defined by the its length divided by its diameter.

The membrane 24 depicted in FIG. 2A is a membrane 24 made of stainless steel. The through-holes 26 therein are created by laser drilling. Laser drilling leads to through-holes 26 having a conical shape with small taper angle. The through-holes 26 obtained in this way have a comparably large aspect ratio AR. The aspect ratio AR of these through-holes may be approximated by the quotient H/d, wherein H is the thickness of the membrane 24 and d is the minimum diameter of the through-hole 26 at the exit end of the membrane 24.

The membrane 24 depicted in FIG. 2B is a membrane 24 made of a nickel-cobalt alloy. The through-holes 26 therein are created by lithography and subsequent electroplating. In a first step lithography is used to create in the membrane 26 a pattern of a plurality of through-holes 26 with a comparatively large diameter. Subsequently, the entire membrane 24 undergoes electroplating to deposit material on the membrane surface thereby shrinking the diameter of the through-holes 26. This results in through-holes 26 having a funnel-like shape. These through-holes 26 have a somewhat smaller aspect ratio AR and would thus be preferred over laser drilled through-holes 26.

The membrane 24 depicted in FIG. 2C is a membrane 24 according to the present disclosure. The membrane 24 comprises a recess 34 in which the through-hole 26 is provided. The remaining thickness h of the membrane 24 in the recess 34 is considerably smaller than the overall thickness H of the membrane 24. Accordingly, the aspect ratio AR of the through-holes 26 of such membrane 24 may be expressed as the quotient h/d, which is much smaller than the aspect ratios AR of the through-holes 26 of the membranes 24 depicted in FIGS. 2A and 2B.

In FIG. 3 a portion of a membrane 24 according to the present disclosure is depicted in more detail. FIG. 3A shows an enlarged view of a recess 34 of such membrane 24. The membrane 24 is made from silicon and has an overall thickness H. The recess 34 has a circular cross section with a diameter D and a depth t. The thickness h of the recessed portion of the membrane 24 is determined as h =H−t. A plurality of through-holes 26 is provided at the recesses 34. These through-holes 26 also have a circular cross section and have a diameter d which is much smaller than the diameter D of the recesses 34.

As indicated in FIG. 3B a membrane 24 may comprise a plurality of recesses 34 while each of the recesses 34 in turn may comprise a plurality of through-holes 26 through which the liquid substrate 14 is released into a mouthpiece chamber 28 of the aerosol-generating device 10.

FIG. 3C shows an electron-microscopical image of a portion of the membrane comprising a recess 34. The membrane has a thickness of about 100 micrometers. The circular recess 34 has a diameter D of about 50 micrometers. The remaining thickness h of the recess 34 amounts to 1 micrometer. The recess comprises 14 circular through-holes 26 which are evenly distributed in the recess 34 in a hexagonal pattern. The diameter d of the through-holes 26 amounts to about 2 micrometers. Thus, the aspect ratio of the through-holes of the membrane shown in FIG. 3C amounts to about 0.5.

A membrane 24 as depicted in FIG. 3 may be manufactured by a sequence of manufacturing steps involving various MEMS processing techniques. A suitable manufacturing method is illustrated in FIG. 4. In a first step a bulk wafer 40 made of silicon is provided as a base substrate. On top of a first surface of the bulk wafer 40 two layers 42, 44 of material are formed by thin film deposition. Layer 42 is formed from silicon dioxide and is used as a selective etching stopper layer. On top of layer 42, there is deposited a layer 44, which is formed from silicon nitride. Layer 44 serves as cover layer of the membrane 24.

In a second step, a mask defining the locations and the diameters of the through-holes 26 is applied to the cover layer 44 by lithography. The cover layer 44 is then dry etched with carbon tetrafluoride (CF 4) to locally remove material of the cover layer 44 and to create through-holes 26 in the silicon nitride cover layer 44.

In a third step, recesses are defined in the second surface or the back side of the silicon bulk wafer 40. For this purpose, a mask defining the position and the cross section of the recesses is applied by lithography to the second surface of the bulk wafer 40. Subsequently, the recesses 34 are formed by etching the second surface of the silicon bulk wafer 40 with sulfur hexafluoride (SF6).

In a fourth step the complete second surface of the silicon bulk wafer 40 is etched further until the bulk waver material is removed from the recesses 34. Due to the use of the selective etching stopper layer 42 made from silicon dioxide, which does not dissolve upon contact with sulfur hexafluoride, etching of the recesses 34 stops once, the silicon dioxide layer 42 is reached. In this step, the bulk wafer material surrounding the recesses 34 is subjected to etching until the bulk wafer has reached a desired thickness. The thickness of the remaining silicon bulk wafer material surrounding the recesses 34 defines the final thickness of the membrane 24.

In a fifth step, the silicon dioxide layer 42 is removed by etching with hydrogen fluoride (HF). By this etching step the accessible portions of the silicon dioxide layer 42 in the recesses 34 are removed, whereby the through-holes 26 in the cover layer 44 are connected to the recesses 34 in the bulk wafer 40. In a last step (not shown) the membrane 24 may be cut out in the desired size from the bulk wafer 40.

With the method illustrated in FIG. 4 a membrane 24 comprising through-holes 26 is obtained by making use of a sequence of MEMS techniques. With these techniques a membrane 24 is obtained that comprises well-defined through-holes 26 having diameters that are significantly smaller than those obtainable by currently used manufacturing techniques. The membrane 24 allows for sufficient liquid throughput and a has a flow resistance that makes the membrane 24 suitable for use in a vibrating mesh module 20 for aerosol-generating devices 10.

In FIG. 5 a further membrane 24 is depicted in which an additional layer of silicon 46 is arranged between two selective etching stopper layers 42A, 42B. The membrane 24 is generally obtained by a similar method as described with FIG. 4 with the exception that in the first manufacturing step a sequence of selective etching stopper layer 42A, silicon layer 46, selective etching stopper layer 42B and cover layer 44 is deposited on the silicon bulk wafer 40 as depicted in the top view of FIG. 5. The final structure of the membrane 24 is depicted in the lower view of FIG. 5. The thickness of the actual membrane 24 is now defined by the additional silicon layer 46 and the recesses 34 are formed in this silicon layer 46. The bulk wafer 40 is largely etched away and only side walls 41 remain as lateral frame structures supporting the membrane 24 extending therebetween. These frame structures and the recesses 34 are again obtained by sequences of masking and etching as described above. In FIG. 5 only two recesses 34 are shown, whereas a membrane 24 may of course comprise a much higher number of recesses 34. In the same way as described with FIG. 4, each recess 34 is again covered by cover layer 44. The cover layer 44 comprises again through-holes 26 through which the liquid substrate 14 is sprayed into the mouthpiece chamber 28 of the aerosol-generating device 10. By using the additional silicon layer 46 between the two selective etching stopper layers 42 A, B a membrane 24 with a well-defined thickness is obtained. Thus, this method allows for a more precise control of the membrane thickness.

By an appropriate choice of materials, the membrane structure may be configured to have desired surface properties that may be beneficial for the aerosolization process. In this regard, FIG. 6 depicts a membrane structure in which an additional layer 48 of polycrystalline silicon is provided below and directly adjacent to the cover layer 44. In this way the surface of the cover layer 44 which is in contact with the liquid substrate 14 stored in the liquid supply portion 12 is lined with the layer 48 of polycrystalline silicon. Since polycrystalline silicon is more hydrophilic as compared to single crystalline silicon or silicon nitride, microfluidic flow through the through-holes 26 in enhanced.

FIG. 7 shows a further modification of the membrane structure depicted in FIG. 4. The bulk wafer 40 forming the base substrate of the membrane 24 is patterned with an electrically conductive structure 50. Patterning of the membrane material may be carried out by using any suitable doping method known to the skilled person. In the method of FIG. 5 the electrically conductive structures 50 may also be formed during vapor deposition of the material layer forming the membrane 24. As depicted in FIG. 7, the electrically conductive structures 50 extend through parts of the membrane material adjacent to the recesses 34 and through-holes 26, but are not exposed to the liquid substrate 14 flowing through the membrane 24. Such electrically conductive structures 50 may form intrinsic resistive heater elements. By running an electric current through these electrically conductive structures 50, the temperature of the membrane 24 and of the liquid substrate 14 to be aerosolized may be adjusted.

MEMS techniques also allow to deposit further functional layers or components on the membrane structure. In this regard, FIG. 8 shows a yet further modification of the previously described membrane structures. The membrane structure is provided with integrated piezo-elements 52 that are formed on an annular contact area 54 of the membrane structure.

The piezo-electric material, in this case lead zirconate titanite, is deposited on top of the membrane structure during the manufacturing process of the membrane 24. As depicted in FIG. 8 the piezo-electric material is deposited in an annually extending contact area 54, which is located at peripheral region of the membrane structure 24. Deposition of the piezo-electric material is carried out by coating techniques, including a masking step so as to achieve the desired lateral geometry of the piezo-elements 52. Forming the piezo-elements 52 integrally with the membrane structure 24 makes manufacturing of vibrating mesh modules 20 easier, compared to conventional manufacturing methods. In conventional manufacturing methods, separately provided piezo-elements 52 have to be glued to a membrane module, which usually is a cumbersome and fault-prone manufacturing step.

The membrane structure 25 may further be provided with contact portions for electrically contacting the piezo-elements 52 to a controller of the aerosol-generating device 10. Such membrane structure as depicted in FIG. 8, which is additionally provided with one or more integrated piezo-elements 52 and electrical contact portions may also be referred to as a vibrating mesh module 20. Such vibrating mesh modules 20 may be used in manufacture of vibrating mesh aerosol-generating devices 10. The vibrating mesh modules 20 may be manufactured as interchangeable accessory of such aerosol-generating devices 10 and may be configured to be exchanged and replaced by the users themselves.

FIG. 9 shows a vibrating mesh module 20 of FIG. 8 connected to an upstream liquid flow chamber 60. The liquid flow chamber 60 is configured to provide liquid substrate 14 to the entrance side of the membrane structure 24. The liquid flow chamber 60 is in fluid communication with a liquid supply portion (not shown) of the aerosol-generating device. The fluid communication is established via an inlet 62 and an outlet 64 which allows the liquid substrate 14 to freely circulate between the flow chamber 60 and the liquid supply portion. This configuration ensures that the entrance side of the membrane 24 is always in contact with the liquid substrate 14. This configuration further ensures that excess liquid substrate 14 that is not dispersed through the through-holes, is fed back into the liquid supply portion. With this configuration the liquid substrate 14 is not pressed against or through the through-holes of the membrane 24. This configuration allows for enhanced reproducibility of the aerosol formation and avoids unwanted leakage of liquid substrate 14.

FIG. 10 shows the embodiment of FIG. 9 connected to the liquid supply portion 66 of an aerosol-generating device. The liquid supply portion 66 holds a supply of liquid substrate 14. The liquid substrate 14 is pumped via pumping device 68 along tubing 70 towards the inlet 62 of the liquid flow chamber 60. A portion of the liquid substrate 14 is dispensed through the membrane and is formed into an inhalable aerosol. Excess liquid substrate 14 that is pumped into the flow chamber 60, which is not dispensed through the membrane 24, may exit the flow chamber via the outlet 64 and is fed back via tubing 72 to the liquid supply portion 66.

1.-13. (canceled)

14. An aerosol-generating device comprising a vibrating mesh module for an aerosol-generating device, the vibrating mesh module comprising:
a MEMS membrane with a plurality of through-holes defining perforations of the MEMS membrane;
a flow chamber located adjacent to the MEMS membrane,
wherein the flow chamber defines a volume configured to hold an aerosol-forming substrate and to feed the aerosol-forming substrate to the MEMS membrane,
wherein the flow chamber is configured to comprise
an inlet through which the aerosol-forming substrate flows into the volume of flow chamber, and
an outlet through which the aerosol-forming substrate may exit the volume of the flow chamber,
wherein the flow chamber is manufactured using MEMS techniques;
a liquid storage portion in fluid communication with the inlet and the outlet of the flow chamber of the vibrating mesh module; and
a pumping device configured to generate a liquid flow through the integrated cavity.

15. The aerosol-generating device according to claim 14,
wherein the perforated MEMS membrane comprises an entrance side, which faces towards the flow chamber, and
wherein the perforated MEMS membrane comprises a release side, through which the aerosol-forming substrate is released into an aerosolization chamber of the aerosol-generating device.

16. The aerosol-generating device according to claim 15, wherein the flow chamber is further configured such that the aerosol-forming substrate flows through the volume of the flow chamber and in contact with the entrance side of the MEMS membrane.

17. The aerosol-generating device according to claim 14, wherein the outlet of the flow chamber is configured to have a flow resistance that is substantially smaller than a flow resistance through the through-holes of the MEMS membrane.

18. The aerosol-generating device according to claim 14, further comprising a piezo-electric actuator configured to produce vibrations of the MEMS membrane.

19. A method of manufacturing a vibrating mesh module for an aerosol-generating device, the method comprising:
manufacturing a membrane from a bulk wafer of a first material using MEMS techniques;
manufacturing a flow chamber from a bulk wafer of a second material using MEMS techniques; and
attaching the flow chamber to the membrane to form the vibrating mesh module,
wherein the flow chamber is configured to comprise
an inlet through which the aerosol-forming substrate flows into the volume of the flow chamber, and
an outlet through which the aerosol-forming substrate may exit the volume of the flow chamber.

20. The method according to claim 19, wherein the bulk wafer material of the membrane is a same material as the bulk wafer used in manufacture of the flow chamber.

21. The method according to claim 19, wherein the membrane and the flow chamber are attached to each other by gluing.

22. The method according to claim 19, wherein the manufacturing of the membrane comprises the steps of:
providing a bulk wafer of a first material,
depositing a cover layer of a second material onto the bulk wafer,
providing through-holes to the cover layer using MEMS manufacturing techniques,
etching the bulk wafer to define recesses therein,
etching the bulk wafer until the bulk wafer material is removed from the recesses, and
cutting out the membrane.

23. The method according to claim 19, wherein one or more piezo-elements are integrally formed on the MEMS membrane.

24. The method according to claim 23, wherein the piezo elements are deposited on the contact surface of the membrane structure by sputtering or by coating techniques.
